(19) United States
(12) Patent Application Publication
(10) Pub. No.: US 2019/0125448 A1
(43) Pub. Date: May 2, 2019

(54) SYSTEM AND METHOD FOR APPLYING CONTROLLED DOSAGE LIGHT THERAPY FOR TREATMENT OF BODY TISSUE

(71) Applicant: RALPH ZIPPER, MELBOURNE, FL (US)

(72) Inventor: RALPH ZIPPER, MELBOURNE, FL (US)

(21) Appl. No.: 16/171,349

(22) Filed: Oct. 25, 2018

Related U.S. Application Data
(63) Continuation-in-part of application No. PCT/US17/21273, filed on Mar. 8, 2017.
Application No. PCT/US17/21273 is a continuation-in-part of application No. 12/687,991, filed on Jan. 15, 2010.
Application No. 12/687,991 is a continuation-in-part of application No. 12/496,216, filed on Jul. 1, 2009, now Pat. No. 8,795,264.
Application No. 12/496,216 is a continuation-in-part of application No. 15/452,958, filed on Mar. 8, 2017.
Application No. 15/452,958 is a continuation-in-part of application No. 12/687,991, filed on Jan. 15, 2010.
Application No. 12/687,991 is a continuation-in-part of application No. 12/496,216, filed on Jul. 1, 2009, now Pat. No. 8,795,264.
Application No. 12/496,216 is a continuation-in-part of application No. 12/687,991, filed on Jan. 15, 2010.
Application No. 12/687,991 is a continuation-in-part of application No. 12/496,216, filed on Jul. 1, 2009, now Pat. No. 8,795,264.
(60) Provisional application No. 61/077,348, filed on Jul. 1, 2008.
(60) Provisional application No. 61/077,348, filed on Jul. 1, 2008.
(60) Provisional application No. 61/077,348, filed on Jul. 1, 2008.

(51) Int. Cl. A61B 18/24; A61N 5/06

(52) U.S. Cl. CPC A61B 18/24; A61B 17/42; A61N 5/0601

(57) ABSTRACT

A method for treating pelvic pain and or chronic prostatitis, and or overactive bladder symptoms. Energy, preferably in the form of infrared or near infrared wavelength light may be applied across the vaginal tissue or rectal tissue to treat pelvic pain and or chronic prostatitis. This method of energy application may cause local heating, alteration of cellular respiration, and alterations of local blood flow resulting in decreased muscle spasm, and or decreased pain, and or decreased overactive bladder symptoms. The method of the present invention may effectively treat chronic pelvic pain and or the symptoms of chronic prostatitis. The method of the invention may combine massaging of tissue with irradiation of same.

### CROSS REFERENCE TO RELATED APPLICATIONS AND INCORPORATION BY REFERENCE

This non provisional patent application is a continuation in part (CIP) application of patent application PCT/US17/21273, filed in the U.S. Receiving Office under the Patent Cooperation Treaty (PCT) on Mar. 8, 2017, which is hereby incorporated in its entirety. This non provisional patent application is also a continuation in part (CIP) application of patent application Ser. No. 15/452,958, filed in the United States Patent and Trademark Office (USPTO) Mar. 8, 2017, which is hereby incorporated in its entirety. Both Ser. No. 15/452,958 and PCT/US17/21273 are CIP applications of U.S. patent application Ser. No. 12/687,991, filed in the USPTO on Jan. 15, 2010, which is also hereby incorporated by reference in its entirety and which was a continuation in part of U.S. patent application Ser. No. 12/496,216, filed in the USPTO on Jul. 1, 2009, which is also hereby incorporated by reference in its entirety, which was a non-provisional of provisional patent application Ser. No. 61/077,348, filed with the USPTO on Jul. 1, 2008, which is herein incorporated by reference in its entirety; and this non provisional patent application is also a continuation in part of U.S. patent application Ser. No. 12/687,991, filed in the USPTO on Jan. 15, 2010, which is hereby incorporated by reference in its entirety and which was a continuation in part of U.S. patent application Ser. No. 12/496,216, filed in the USPTO on Jul. 1, 2009, which is also hereby incorporated by reference in its entirety and which issued as U.S. Pat. No. 8,795,264, which was a non-provisional of provisional patent application Ser. No. 61/077,348, filed with the USPTO on Jul. 1, 2008, which is herein incorporated by reference in its entirety. U.S. non-provisional patent application Ser. No. 12/687,965 which published as US 2011-0004203 A1 on Jan. 6, 2011 is also incorporated herein by reference in its entirety.

### STATEMENT REGARDING FEDERALLY SPONSORED RESEARCH OR DEVELOPMENT

Not applicable.

### INCORPORATION-BY-REFERENCE OF MATERIAL SUBMITTED ON A COMPACT DISK

Not applicable.

### BACKGROUND OF THE INVENTION

### 1. Field of the Invention

The present invention generally relates to a method of applying laser energy as a treatment regimen to body tissue, which may be human or non-human body tissue. In a non-limiting embodiment, the present invention relates to a system and method for applying laser energy to vaginal and or rectal tissue to treat the vagina and rectum generally, including but not limited to treating the causes and symptoms of chronic pelvic pain, chronic prostatitis, and overactive bladder.

### 2. Background Art

Pelvic disorders such as, for example and not by way of limitation, chronic pelvic pain and chronic prostatitis are highly prevalent and highly debilitating. Over 50 million U.S. women and over 100 million women worldwide suffer from chronic pelvic pain. In the absence of readily available and effective treatments, only 40% of chronic pelvic pain patients are referred to specialists. Over 8 percent of men suffer from chronic prostatitis. This is a prevalence similar to diabetes and heart disease. Similar to chronic pelvic pain, there are few effective treatments. Up to 90% of men with chronic prostatitis meet the criteria for chronic nonbacterial prostatitis/chronic pelvic pain syndrome, a disorder with few treatment options supported by only limited evidence. Although approximately 16% of men and women suffer from the symptoms of overactive Bladder, multiple treatments are available. Existing treatments for overactive Bladder include prescription medications taken in perpetuity, with numerous side effects, repetitive injections of botulinum toxin with associated side effects and only transient improvement, implantable neuromodulation devices costing fifteen to twenty thousand dollars per implant, and repetitive percutaneous nerve stimulation. There are no non-systemic, non-invasive treatments for overactive bladder symptoms.

What is needed in the art, therefore, is a non-invasive method for treating the above identified conditions that is safe and effective.

### BRIEF SUMMARY OF THE INVENTION

The present invention comprises an apparatus and method that have one or more of the following features and/or steps, which alone or in any combination may comprise patentable subject matter.

The present invention overcomes the shortcomings of the prior art in that it provides a non-invasive method for treating the above identified conditions that is safe and effective.

In accordance with one embodiment, a method for treating pelvic pain and or chronic prostatitis, and or overactive bladder symptoms by way of the trans-tissue transmission of laser energy, preferably in the infrared or near infrared spectrum, wherein the method comprises the steps of providing a probe capable of emitting said energy to and through the vaginal and or rectal tissue, inserting said probe into the vagina and or rectum, and activating the source of the energy. In the preferred embodiment, the probe is thence kept in continuous, back and forth, motion until the appropriate dose of energy has been administered.

In accordance with an embodiment of invention, a method and system for applying specific doses of light energy, wherein the method and system control the amount of energy delivered to the treated tissue or the density of the energy delivered to the treated tissue, or both, by using markings on the probe in conjunction with visual, auditory or vibrotactile cues to the treating physician, or operator, of the system of the invention. The total amount of energy, the density of the energy delivered to the treated tissue, or both, are controlled to achieve maximum therapeutic effect on the patient.

### BRIEF DESCRIPTION OF THE DRAWINGS

The accompanying drawings, which are incorporated into and form a part of the specification, illustrate one or more embodiments of the present invention and, together with the description, serve to explain the principles of the invention. The drawings are only for the purpose of illustrating the preferred embodiments of the invention and are not to be construed as limiting the invention. In the drawings:

FIG. 1 depicts an exemplary flow chart for the first embodiment of the method of the invention for treating the vagina of a patient.

FIG. 2 depicts a laser source and probe system such as may be utilized in carrying out the steps of the invention.

FIG. 3A depicts a probe apparatus such as may be utilized in carrying out the steps of the invention, depicting a probe with a bulbous or spherical massaging light emitting portion and depicting an exemplary radiation pattern. FIG. 3B depicts an exemplary cross section of the probe depicted in FIG. 3A.

FIGS. 4A and 4B depicts a conical embodiment of probe apparatus such as may be utilized in carrying out the steps of the invention.

FIGS. 5A and 5B depicts a trapezoidal embodiment of probe apparatus such as may be utilized in carrying out the steps of the invention.

FIG. 6 depicts a generalized probe apparatus such as may be utilized in carrying out the steps of the invention for use in treating vaginal tissue, depicting energy radiating from the probe in a direction along a longitudinal axis of the probe in a spherical or partially spherical pattern.

FIG. 7 depicts a probe apparatus such as may be utilized in carrying out the steps of the invention, depicting the probe inserted just beyond the opening of the vagina of a patient, and showing laser energy emitted from the probe. FIG. 6 Further depicts a probe apparatus such as may be utilized in carrying out the steps of the invention, depicting the probe inserted into the vagina of a patient, depicting the vaginal tissue conforming to the bulbous tip of the probe, and depicting the total distance of travel L of the laser probe within the vagina for an embodiment of the method of the invention.

FIGS. 8A and 8B depict a generalized probe apparatus such as may be utilized in carrying out the steps of the invention for use in treating the prostate, depicting energy radiating from a side of the probe in a direction transverse to a longitudinal axis of the probe so as to illuminate the length of the tissue overlying the prostate. FIG. 8A depicts a side view, and FIG. 8B depicts an end view.

FIG. 9 depicts a probe apparatus such as may be utilized in carrying out the steps of the invention for use in treating the prostate, depicting the probe in relation to the rectum walls, and depicting the rectum walls conforming around the bulbous tip of the probe. FIG. 9 further depicts the probe inserted into the rectum at the point to the point where the first (proximal) mark is located at the anus, at which point the laser energy source may be activated and deactivated. FIG. 9 also depicts and further depicts the probe inserted into the rectum to the point where the second (distal) mark is located at the anus marks on the probe.

FIG. 10 depicts an exemplary pulsatile waveform of an embodiment of the method of the invention.

FIG. 11 depicts an embodiment of a probe of the invention, showing embodiment of a temperature sensor disposed on the probe for measuring the temperature of body tissue.

FIG. 12a depicts an embodiment of a probe of the invention for vaginal use, in which the massaging portion and the light emitting portion are separated by a distance Z.

FIG. 12b depicts an embodiment of a probe of the invention for rectal use, in which the massaging portion and the light emitting portion are separated by a distance Z.

FIG. 12c depicts an embodiment of handle 002 in which marks 500 are disposed on probe, and further depicting the probe inserted into vaginal tissue. This figure further depicts the treatment surface area for an embodiment of the invention. This figure further provides definition of the proximal and distal reference, and defines IN as moving in the distal direction and OUT as moving in the proximal direction.

FIG. 13 depicts a block diagram of an embodiment of the temperature probe and laser source controller of the invention.

FIG. 14 depicts a block diagram of an embodiment of the invention in which temperature sensor 301 is in communication with controller 350 which is in communication with optional video display 510, speaker 514, foot switch 513 and haptic actuator 515.

FIG. 15 depicts a flow diagram of an embodiment of a method of the invention.

In the figures, like callout numbers refer to like elements.

### DETAILED DESCRIPTION OF THE INVENTION

The following documentation provides a detailed description of the invention.

Although a detailed description as provided in the attachments contains many specifics for the purposes of illustration, anyone of ordinary skill in the art will appreciate that many variations and alterations to the following details are within the scope of the invention. Accordingly, the following preferred embodiments of the invention are set forth without any loss of generality to, and without imposing limitations upon, the claimed invention. Thus the scope of the invention should be determined by the appended claims and their legal equivalents, and not merely by the preferred examples or embodiments given.

As used herein, “memory”, “medium”, “media”, “computer readable memory”, “computer readable medium”, “storage media”, “computer readable storage media” and “computer readable storage medium” shall include within their meanings only physical non-transitory computer readable hardware, and such terms shall specifically exclude signals per se, carrier waves, propagating signals and other transitory signals. Such physical non transitory computer readable media may comprise hardware memory that comprises a physical structure for storing data which may include computer executable instructions or data.

As used herein, “controller” includes within its meaning any electrical device or combination of electrical devices capable of executing computer readable instructions such as a controller, processor, microcontroller, microprocessor, field programmable gate array, programmable logic array, embedded firmware, virtual machine, combinational logic or any other electrical or electronic device or any combination of devices known in the electrical arts as capable of executing computer readable or hardcoded instructions.

As used herein, with respect to the controller, “adapted to” includes within its meaning that the controller executes computer readable non-transitory or hardcoded instructions, or utilizes hardwired logic or any other means known in the art for controlling a feature, device, or signal, to carry out the subject function. Any function herein described as being caused or commanded by the controller of the invention may be carried out by the controller executing computer readable non-transitory instructions, or utilizing hardwired logic, programmable logic such as gate arrays, or any other means known in the art for controlling a feature, device, or signal, to carry out the subject function

As used herein, “near infrared” means optical wavelengths between 700 nm and 1,400 nm.

As used herein, “IN and OUT” has the same meaning as “back and forth”, where “IN” corresponds to “forth” and “OUT” corresponds to “back”.

As used herein, “potential space” means the space that, prior to insertion of a probe of the invention into such space, may have no, or a small; volume which is unoccupied by body tissue, but which, when a probe of the invention is inserted into the potential space, the body tissue comprising the surface of the potential space is displaced and stretched around the probe as the probe is inserted into and translated through the potential space.

As used herein, “massage”, “massaging” or “massaging effect” is the effect on the tissue comprising a potential space resulting from the body tissue being displaced around the outer surface of a probe of the invention.

As used herein, “in communication with” has the same meaning as “operatively coupled”.

Power and energy density measurements are referenced to the surface of the probe light emitting portion 101 (or 101a, 101b, 101c, etc.). In the case which the power or energy density is not uniform across the surface that is emitting the laser energy, the power or energy density given is an average power density as measured across the total surface area emitting the laser energy.

The scope and breadth of the present inventive disclosure is applicable across a wide variety of procedures, tissues and anatomical structures. Although the following detailed description contains many specifics for the purposes of illustration, anyone of ordinary skill in the art will appreciate that many variations and alterations to the following details are within the scope of the invention. The method of the invention may be applied to any tissue, and thus is not limited to rectal or vaginal tissue only. Accordingly, the following preferred embodiments of the invention are set forth without any loss of generality to, and without imposing limitations upon, the claimed invention.

Each of the preferred embodiments of the treatment method of the invention may share the clinically effective novel dosing regimen and parameters of the invention that were discovered and developed only through the extensive experimentation by the inventor, including multiple clinical trials. The experimentation led to a determination of the specific claimed treatment parameters that were observed to be efficacious when laser energy was applied to body tissue using a probe in the manner described herein. These novel dosing and treatment parameters comprise the administration of light energy at of 4-10 watts for vaginal use and 2-8 watts for rectal use; the administration of between 2000 and 4000 joules of near infrared light per treatment; the administration of such energy at a power density between 0.09 W/cm² and 6.0 W/cm² measured at the exterior surface of the light emitting portion of the laser probe; and the movement of the probe along the treatment area at a rate between 2.5 millimeters per second and 60 millimeters per second to provide both a massaging and an irradiating effect. Ideally, the power density (W/cm²) to probe speed ratio shall (mm/sec) was experimentally determined to be between 0.0042 and 1.8. Outside this range, therapeutic effect was lost.

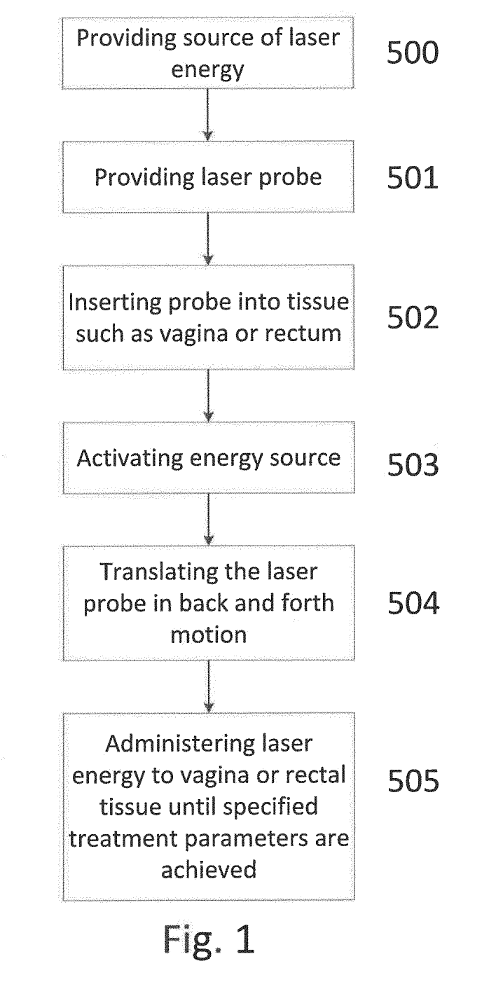

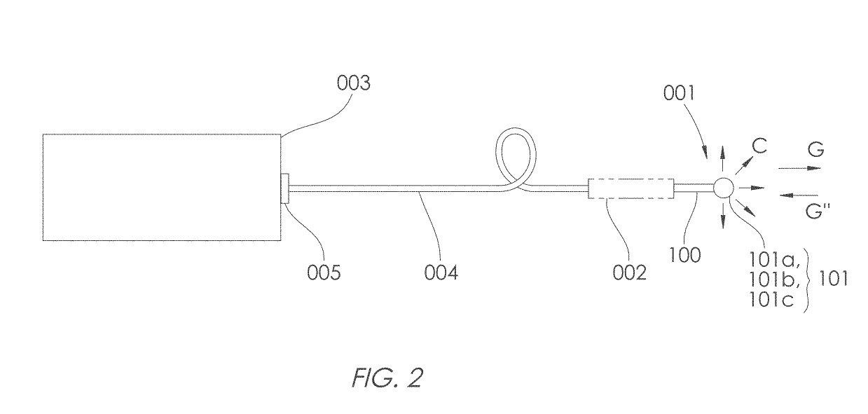

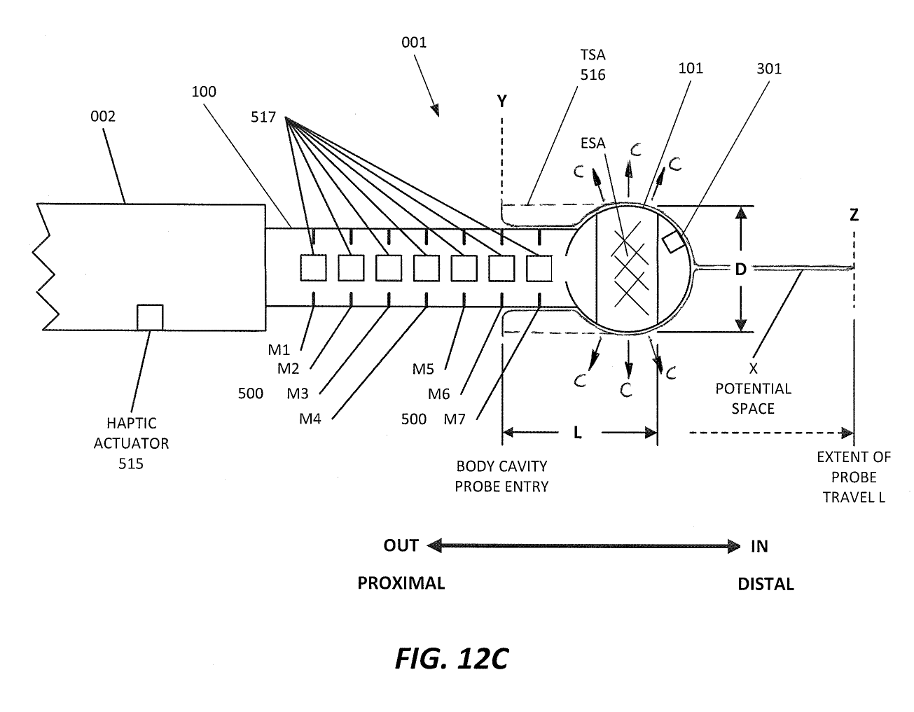

Referring now to FIGS. 1 and 2, the method of the invention comprises, generally, the steps of providing a source of laser energy 500, providing a probe 001 capable of emitting laser energy to body tissue such as vaginal tissue or rectal tissue, probe 001 being in optical communication with said source of laser energy 003 via optical fiber or cable 004, and probe body portion 100 which attaches probe portion 101 to probe handle 002. Handle 002 may be any apparatus that is adapted to advance or retract, or both, probe body portion 100 in the direction of arrows G and G′ which are oriented along an axis of the probe. Likewise, although specific embodiments of probe 001 are described herein, probe 001, which comprises a probe body portion 100 and a probe massaging portion 101, may be any probe capable of emitting laser energy at near infrared wavelengths. In an embodiment, probe massaging portion 101 may be the portion of the probe that emits light energy. In other embodiments in which the probe massaging portion 101 and the portion of the probe that emits light energy are different features, it is preferred, but not necessary, that these features be located along the probe within one centimeter of each other so that the illumination of body tissue with near infrared light from the light emitting portion of the probe is simultaneously, or nearly simultaneously, receiving a massaging effect by the translation of the probe massaging portion 101 along the treated tissue. In other embodiments, the light emitting portion is defined as being a separate structure from the massaging portion such as depicted in FIGS. 12A and 12B.

Still referring to FIG. 1, the method further comprises the step of inserting the probe into body tissue such as the vagina or rectum of a person, who may be a patient receiving treatment, 502, such that at least a portion of the probe is in physical contact with a portion of said body tissue, followed by the step of activating the source of laser energy 503. The probe may then be translated according various embodiments of the invention to provide a massaging effect on the vaginal or rectal tissue 504, or until a specific treatment parameter has been reached, 505, or both.

Referring now to FIG. 2, a laser source and probe system such as may be utilized in carrying out the steps of the invention is depicted.

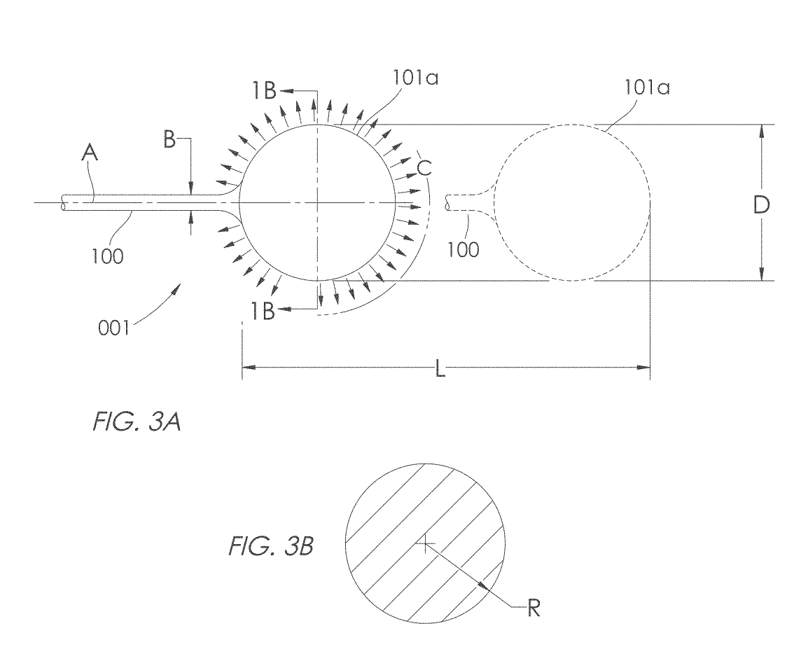

The probe may, by way of example and not by limitation, be laser probes such as the exemplary embodiments of probes depicted in FIGS. 3A, 3B, 4A, 4B, 5A and 5B. While these exemplary shapes are depicted, it is understood that the scope of the invention includes any shape for which a massaging portion 101 of the probe is of greater outer dimension than probe body portion 100.

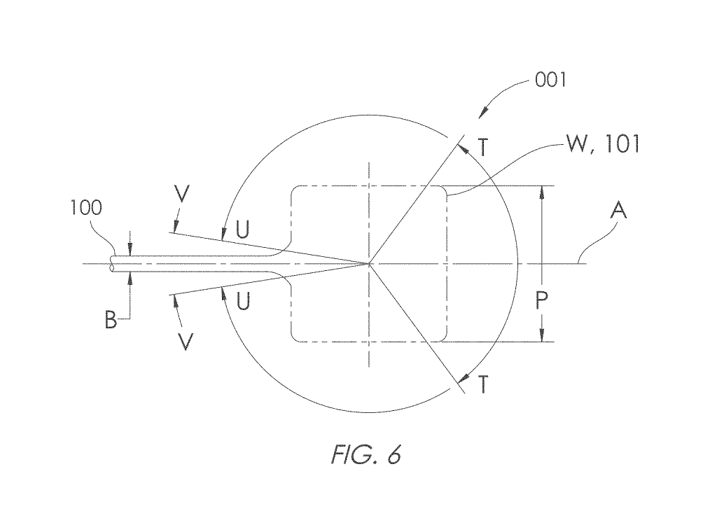

Referring now to FIGS. 3A and 3B, a spherical embodiment of a probe 001 that may be used to carry out the method of the invention is depicted. Probe body portion 100 has outer dimension B and communicates laser energy from laser source 003 (not shown in FIGS. 3A and 3B, but depicted in FIG. 2) to probe light emitting portion 101, which is, in the embodiment depicted, a spherical or bulbous shape of radius R. During use in a method of the invention, probe 001 may be translated a distance L, measured as depicted in the figure. Light energy C may be projected outwards from massaging portion 101 in a substantially or partially spherical pattern, except for the area of the sphere attached to probe connector 100 which may be of outer dimension B as is further discussed in relation to FIG. 6 below.

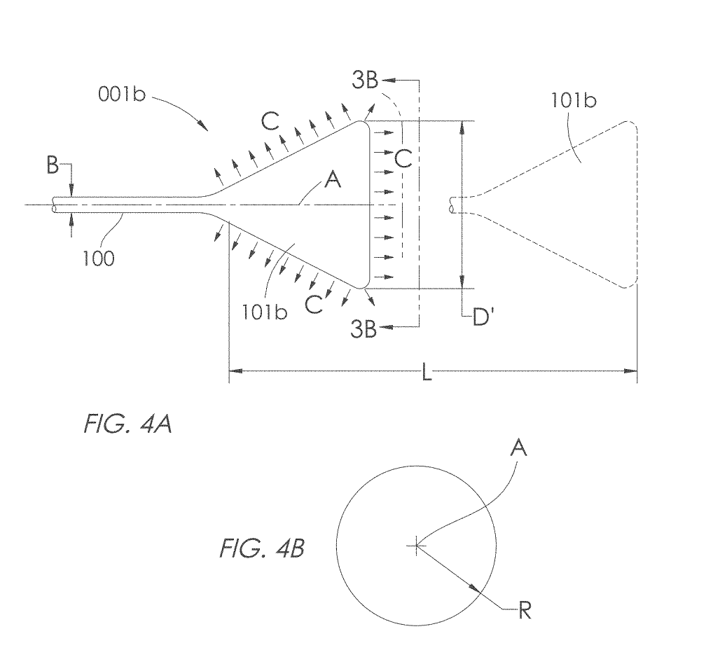

Referring now to FIGS. 4A and 4B, a conical embodiment of a probe 001 that may be used to carry out the method of the invention is depicted. Probe body portion 100 has outer dimension B and communicates laser energy from laser source 003 (not shown in FIGS. 4A and 4B, but depicted in FIG. 2) to probe light emitting portion 101, which is, in the embodiment depicted, a conical shape having an end face of radius R. During use in a method of the invention, probe 001 may be translated a distance L, measured as depicted in the figure. Light energy C may be projected outwards from massaging portion 101, except for the area of the massaging portion attached to probe body portion 100 which may be of outer dimension B.

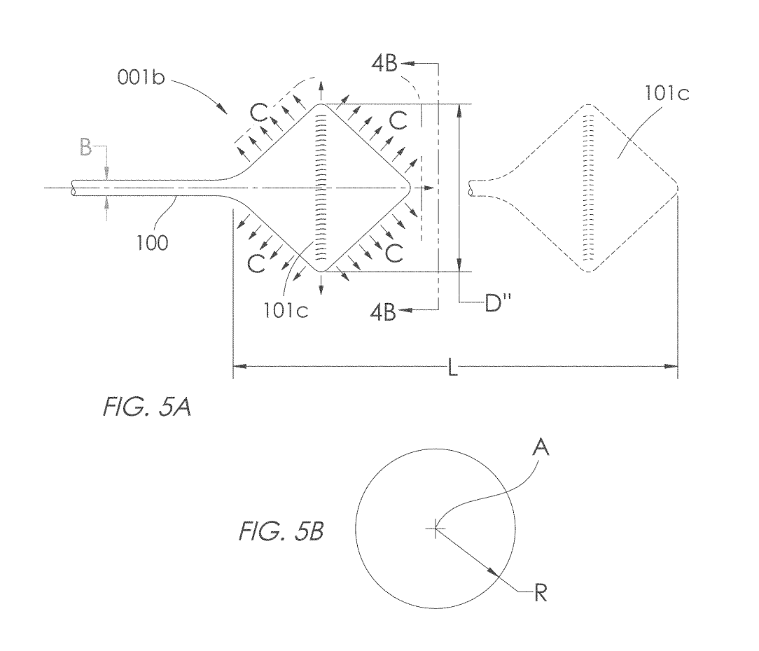

Referring now to FIGS. 5A and 5B, an embodiment of a probe 001 that may be used to carry out the method of the invention is depicted. Probe body portion 100 has outer dimension B and communicates laser energy from laser source 003 (not shown in FIGS. 5A and 5B, but depicted in FIG. 2) to probe light emitting portion 101, which, in the embodiment depicted, has an outer dimension characterized by radius R. During use in a method of the invention, probe 001 may be translated a distance L, measured as depicted in the figure. Light energy C may be projected outwards from massaging portion 101, except for the area of the massaging portion attached to probe body portion 100 which may be of outer dimension B.

In the embodiments depicted in FIGS. 3A, 3B, 4A, 4B, 5A, and 5B, the light emitting portion and the massaging portion may be the same structure 101a, b, or c respectively.

Referring now to FIG. 6, a probe 001 having any outer shape W such as may be utilized in carrying out the steps of the invention for use in treating vaginal tissue, depicting energy radiating in a partially spherical pattern from the probe in a direction along a longitudinal axis of the probe A in a radiation pattern is depicted. In FIG. 6, outer shape W of probe massaging portion 101 is depicted in broken lines to indicate that the probe massaging portion 101 may be spherical, conical, trapezoidal or any three dimensional shape. While spherical, conical, and trapezoidal shapes are described herein as examples of the shape of the massaging portion 101, it is understood that the scope of the invention includes any shape for which outer dimension P is greater than dimension B, i.e., for which the massaging portion of the probe is of greater outer dimension than the probe body portion 100. In this embodiment of the probe, light is emitted in a radiation pattern that ranges from partially spherical as depicted by angle T-T which may be for example 120°, to nearly spherical as depicted by angle U-U, and having an axis that is coaxial with the longitudinal axis A of the probe. In this context, “nearly spherical” means a radiation pattern that is spherical except for the partially spherical angle depicted as V-V in which little or no light may be transmitted due to probe body portion 100 having an outer dimension B. Thus, the value of partially spherical angle V-V is dependent upon the value of B. The radiation pattern thus described causes the treatment of the vaginal walls encountered during energy transmission without the need for rotation of the probe in order to fully illuminate the treated vaginal walls. In yet another preferred embodiment, the center of the radiation pattern may be along an axis that is transverse to the longitudinal axis of the probe A. By way of example, in a 180 degree or half sphere radiation pattern along such a transverse axis, treatment could be limited to the rectal or bladder side of the vagina and such side could be treated without rotation of the probe.

### Vaginal Treatment

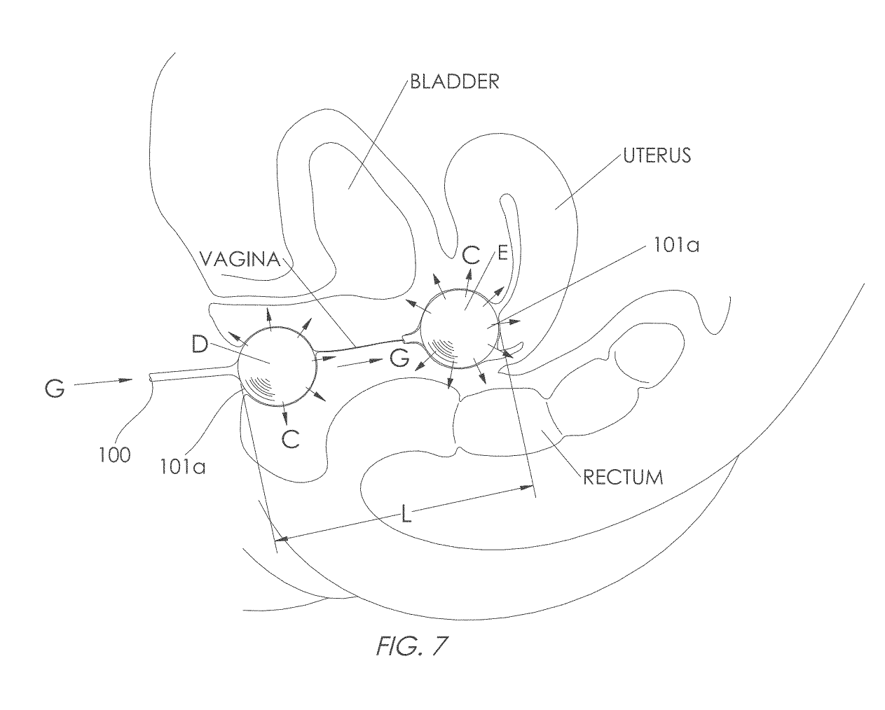

Referring now to FIG. 7, in an embodiment, the method of the invention is directed to treating vaginal tissue. In this embodiment, the laser probe massaging portion 101 (depicted for example as a spherical massaging portion 101a) is inserted into a vagina in the direction of arrow G, the source of laser energy is activated such that either continuous wave or pulsatile laser energy is transmitted from the laser source, through the probe body portion 100, and into the surrounding vaginal tissue thus treating this tissue. The laser probe massaging portion 101 may then may be translated along the probe longitudinal axis A (not shown in FIG. 6 but shown in FIG. 3A), in a back and forth fashion, over a treatment length L between proximal position D and distal position E, until specific experimentally derived treatment parameters for the delivery of laser energy to the vaginal tissue surrounding the laser probe are met. Probe body portion 100 is depicted for reference. Certain anatomical structures, including the bladder, rectum and uterus, are also depicted for reference. Although FIG. 6 depicts a probe comprising a spherical or bulbous massaging portion 101a, a probe of any shape may be used.

Still referring to FIG. 7, the back and forth motion of the probe over distance L between positions D and E may be, and is preferably, continuous. Continuous is defined as back and forth movement that is interrupted in the instant of a change in direction when the probe has traversed distance L and is located at either proximal position D or distal position E such as is created by an in and out, back and forth, or IN and OUT movement. FIG. 12C defines “proximal”, “distal, “IN” and “OUT”. This movement of the probe within the vagina creates a mechanical massage effect on the vaginal tissue that is in physical contact with massaging portion 101. This mechanical massage effect is caused by the displacing of tissue around the outer surface of the probe as the probe is translated through the potential space of the vagina. The displacement and stretching of tissue in this manner, preferably but not necessarily occurring simultaneously with the transmission of light energy from the probe, provides an unexpected therapeutic result. Multiple years of clinical investigation has led to the invention of a specific range of treatment parameters. Outside of these treatment parameters, efficacy was lost and or patient discomfort was encountered. These treatment parameters include movement of the probe at a rate between 2.5 millimeters per second and 60 millimeters per second, a total dose of 200-600 joules per linear centimeter of vagina treated or 13.045-310.546 joules/cm², whereas such area is calculated using the linear distance of vagina treated and the radius of the energy emitting portion of the probe, and a power density is between 0.09 W/cm² and 6.0 W/cm². In a preferred embodiment, the diameter of the energy emitting portion of the probe is between 1.5 and 3.5 cm, the probe velocity is between 10 mm/sec and 40 mm/sec, the power density (W/cm²) to probe speed (mm/sec) ratio shall be between 0.0042 and 1.8, total dose is 200-400 joules per linear centimeter or 21.068-149.208 joules/cm2, and the power density between 0.09 W/cm² and 6.0 W/cm². Distance L is defined as the distance between the most proximal and most distal points of treatment. Once the above noted dose has been administered, the energy source is deactivated and the probe is removed from the vagina. If the energy source is capable of recording and or otherwise utilizing the linear distance, such information shall be measured and entered in to the energy source prior to the transmission of laser energy through the probe. With regard to dosing by joules/cm², it is important to understand that the vagina exists as a potential space rather than an open space. Once a probe is placed into the vagina, the potential space is opened, the vaginal tissue conforms around the probe, and the diameter of the space thence becomes the diameter of the inserted probe. This Probe Treatment Surface Area, PTSA, has not been previously described, creates an objective measurement allowing for a reproducible treatment protocol with resultant reproducible safety and efficacy.

In a further embodiment of the method for treating a vagina, the energy is transmitted in a pulsatile or continuous fashion until a total dose of 200-600 joules per linear centimeter or 13.045-310.546 joules/cm² of energy has been transmitted into vaginal tissue, wherein such area is calculated using the linear distance of vagina treated and the radius of the energy emitting portion of the probe, and a power density is between 0.09 W/cm² and 6.0 W/cm². This is the therapeutic range shown to be safe, comfortable, and effective through extensive experimentation. In a preferred embodiment, the diameter of the energy emitting portion of the probe is between 1.5 and 3.5 cm, the power density (W/cm²) to probe speed (mm/sec) ratio is between 0.0042 and 1.8, total dose is 200-400 joules per linear centimeter or 21.068-149.208 joules/cm², and the power density is between 0.09 W/cm2 and 6.0 W/cm². In a preferred embodiment, the probe projects said energy in a 120-360 degree partially spherical to nearly spherical radiation pattern having an axis that is co-axial with probe longitudinal axis A as depicted in FIG. 6 and described above in relation thereto.

In an embodiment, once the probe is inserted in a vagina, the energy source 003 is activated and laser energy is preferably transmitted though the probe massaging portion 101 or light emitting portion 102a as shown in FIG. 12A and into the vaginal tissue surrounding the probe. Distance L is defined as the distance between the most proximal and most distal points of treatment. The laser energy source 003 is then inactivated and the probe is advanced or withdrawn within the vagina by one centimeter. This advancement or withdrawal may be facilitated by markings on probe body portion 100 that are visible from outside the vagina when the probe is inserted into the vagina. This process is repeated until the vaginal canal or painful area of the pelvic floor is fully treated; i.e., until one of the dosage parameters has been met. The energy source is then deactivated and the probe is removed from the vagina.

The vaginal treatment methods of the invention may be facilitated by a group of novel device features. Such features include markings on the probe connector portion 100 to demonstrate distance of insertion and a temperature sensor on the probe which provides feedback to the energy source. In one embodiment, the energy source adjusts pulse shape and/or amplitude and or time between pulses in order to maintain surface temperature between 40 and 45 degrees centigrade. In another embodiment, the energy source shall adjust pulse shape and/or amplitude and or time between pulses in order to maintain surface below 45 degrees centigrade. Auditory and or visual cues may be provided by the energy source user interface and or other hardware to facilitate the movement of the probe at the prescribed rate. Such ques may rely upon the input of the probe diameter or power density information into the energy source and or an accelerometer contained within the probe or handle of the probe. Such cues may also rely upon surface temperature measurements taken by a temperature measuring mechanism within the probe that provides such temperature measurement to the energy source and or accessory hardware. In the preferred embodiment, the probe shall have a variation in outside dimension of any cross sectional shape such that a massage or milking effect is created on the treated tissue. By way of example and not limitation, this alteration in probe outer dimension may take a spherical shape (see FIGS. 3A and 3B), conical shape (see FIGS. 4A and 4B), or trapezoidal shape (see FIGS. 5A and 5B). The shape of the probe massaging portion outer surface 101 may take any three dimensional shape; the example shapes depicted in FIGS. 3A, 3B, 4A, 4B, 5A, and 5B are exemplary and not exhaustive.

### Prostate Treatment

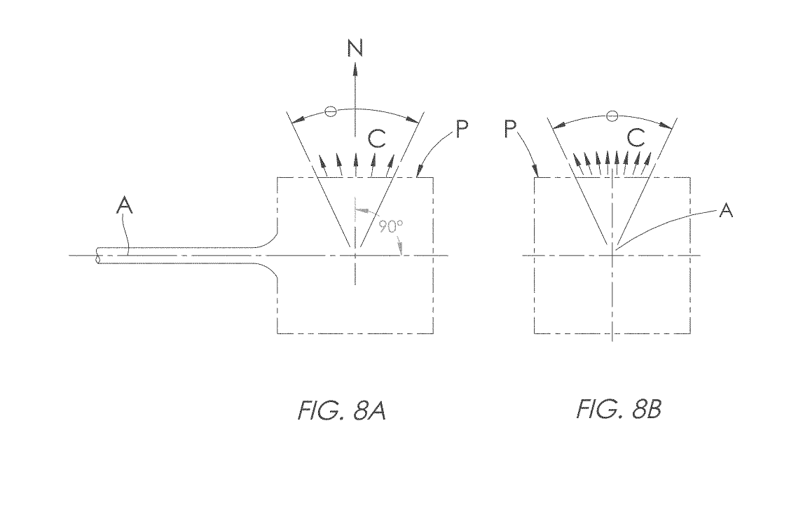

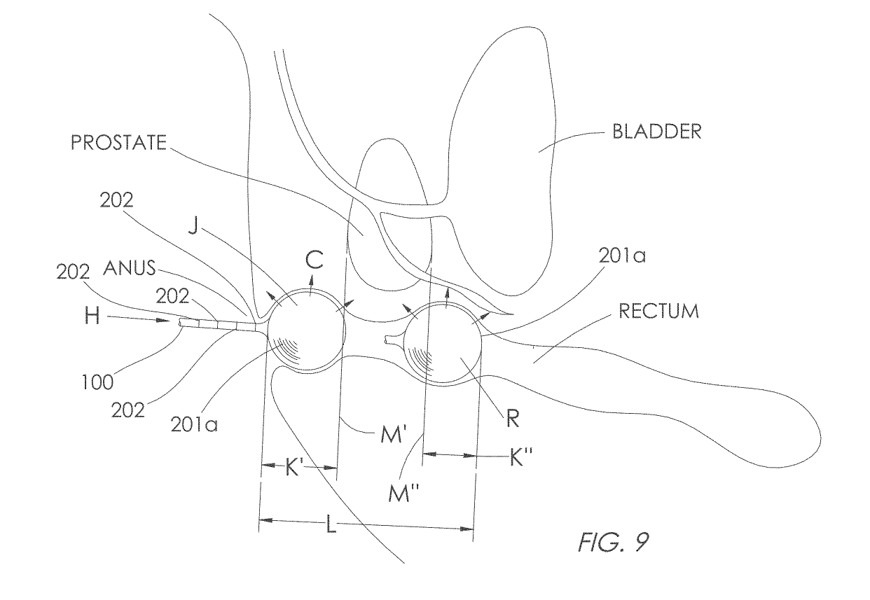

Referring now to FIGS. 8A, 8B, and 9, in a further embodiment, the method of the invention is directed to treating a prostate by irradiating through the rectum wall into the prostate. This embodiment of the invention involves the placement of a probe of the invention attached to an energy source capable of transmitting continuous wave or pulsatile near infrared wavelengths though said probe, into the rectum, by inserting probe massaging portion 201 into the rectum in the direction of arrow G. In a preferred embodiment, the probe is adapted such that it projects said laser energy in a partially spherical radiation pattern having an axis N as shown in FIGS. 8A and 8B in which N is orthogonal to the probe longitudinal axis A, and in which θ is between 30°-180°. Once inserted in the rectum the probe is oriented such that the emitted laser energy is projected predominantly toward the prostate, the energy source is activated and, thence, laser energy is transmitted though the probe and into the surrounding rectal tissue, passing through the rectal tissue and into the prostate. The proper orientation of the probe may be facilitated by a mark on the handle of the probe or a shape of the handle probe that allows the surgeon or other person performing the method of the invention to align the transmitted optical energy from the probe in the direction of the prostate. The probe is then translated in a back and forth motion over a distance L between positions J and R until specified parameters are achieved. This back and forth translation may be performed continuously, with exception for the instant in which direction of movement is reversed. This translation creates a mechanical massage effect by displacing and stretching tissue around the outer surface of the probe as the probe is translated. The displacement and stretching of tissue in this manner as the probe is translated through the potential space of the rectum, preferably but not necessarily occurring simultaneously with the transmission of light energy from the probe, provides an unexpected therapeutic result. Multiple years of clinical investigation led to the invention of a specific range of treatment parameters. Outside of these treatment parameters, efficacy was lost and or patient discomfort was encountered. Treatment parameters include movement of the probe at a rate between 2.5 millimeters per second and 60 millimeters per second, a total dose of 200-600 joules per linear centimeter of rectum treated or 13.045-310.546 joules/cm², whereas such area is calculated using the linear distance of rectum treated and the radius of the energy emitting portion of the probe, and a power density is between 0.09 W/cm² and 6.0 W/cm². In a preferred embodiment, the diameter of the energy emitting portion of the probe shall be between 1.5 and 3.5 cm, the probe speed shall be between 10 mm/sec and 40 mm/sec, the power density shall be between 0.09 W/cm² and 6.0 W/cm², the power density (W/cm²) to probe speed ratio between 0.0042 and 1.8, total dose shall equal 200-400 joules per linear centimeter or 21.068-149.208 joules/cm², and the power density shall be between 0.09 W/cm² and 6.0 W/cm². The distance L is defined as the distance between the most proximal and most distal points of treatment. Once the above noted dose has been administered, the energy source is deactivated and the probe is removed from the rectum. In the preferred embodiment, the linear distance is that distance beginning one centimeter proximal to the prostate and ending one centimeter distal to the prostate. In the preferred embodiment, markings on the probe body portion 100 are used to identify the range of rectum lying beneath and preferably, but not necessarily slightly beyond the margins of the typical prostate and movement occurs between such markings, the linear distance. In an alternative embodiment, the distance between such markings may correspond to the length of the prostate to be treated. If the energy source is capable of recording and or otherwise utilizing the linear distance, such information may be measured and entered in to the energy source prior to the transmission of laser energy through the probe.

In an embodiment of the method of the invention directed to treating a prostate by transmitting laser energy in a pulsatile fashion, such energy may emitted by the massaging portion of the probe until a total dose of 200-600 joules per linear centimeter or 13.045-310.546 joules/cm² of energy has been emitted from the probe, wherein such area is calculated using the linear distance of rectum treated and the radius of the energy emitting portion of the probe, and a power density is between 0.09 W/cm² and 6.0 W/cm². This is the therapeutic range shown to be safe through extensive experimentation. In a preferred embodiment, the diameter of the energy emitting portion of the probe shall be between 1.5 and 3.5 cm, the power density (W/cm²) to probe speed (mm/sec) ratio shall be between 0.0042 and 1.8, total dose shall equal 200-400 joules per linear centimeter or 21.068-149.208 joules/cm², and the power density shall be between 0.09 W/cm² and 6.0 W/cm². Distance L is defined as the distance between the most proximal and most distal points of treatment. The energy source is inactivated and the probe is advanced or withdrawn by one centimeter. This advancement or withdrawal may be facilitated by markings 202 on the probe. This process is repeated until the area overlying the prostate has received a predetermined dose of energy. The energy source is deactivated and the probe is removed from the rectum. Ideally, the pulse shape and time as well as the time between pulses shall be set to maintain a surface below 45 degrees centigrade.

Still referring to FIG. 9, treatment distance L may be defined as extending from between points defined as being disposed beyond a proximal boundary M′ of the prostate and distal boundary of the prostate M″ by distances K′ and K″, respectively. For example, K′ and K″ may each equal one centimeter, and thus the massaging portion may be translated in back and forth fashion along L, treating the prostate and the area that extends on either side of the prostate by one centimeter. Distances K′ and K″ however may take any value.

In an alternative embodiment of the method of the invention, laser energy may be transmitted without translation of the probe over distance L. In this embodiment, the probe is adapted to transmit a wide beam of laser energy that covers the entire distance of tissue for which treatment is desired. In this alternative embodiment, probe body portion 100 is marked having a marking that, when the marking is at the level of the anus and the energy source is activated, laser energy is transmitted in a continuous or pulsatile fashion until the desired dosing is administered; i.e., the desired dosing parameter is met. The energy source is next inactivated and the probe removed.

Any of the embodiments directed to a method for treating a prostate may be facilitated by a group of novel device features. Such features include measurement markings upon the probe body portion. Such measurement marking provide the linear distance treated. Such features include markings on the probe body portion representing the range of rectum overlying the typical prostate and one cm marking or other equidistant markings between these markings, and a marking on the handle and or a handle shape that orients the energy, predominantly, toward the prostate. Ideally, the proximal and distal markings will be such that treatment between said markings results in a linear distance that extends one centimeter distal and one centimeter proximal to the prostate location above the rectum. A marking on the handle of the probe may facilitate orientation of the probe such that energy is transmitted predominantly toward the prostate. Auditory and or visual cues may be provided by the energy source user interface and or other hardware to facilitate the movement of the probe at the prescribed rate. Such cues shall rely upon the input of the probe diameter or power density information into the energy source and or an accelerometer contained within the probe or handle of the probe. Such cues may also rely upon surface temperature measurements taken by a temperature measuring mechanism within the probe that provides such temperature measurement to the energy source or accessory. In the preferred embodiment, the probe massaging portion has a greater outer dimension that the probe body portion such that a massage or milking effect is created. By way of example, the probe massaging portion may comprise a spherical shape, trapezoidal shape, conical shape or any other shape. Ideally but not necessarily, said probe massaging portion is disposed at or within one cm of the energy emitting area of the probe. A temperature sensor on the probe may provide feedback to the energy source.

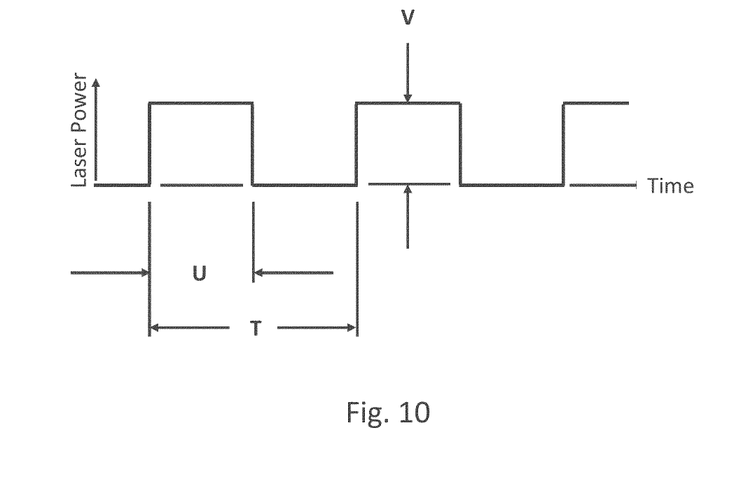

Referring now to FIG. 10, in any embodiment of the method of the invention using pulsatile laser energy, the pulse shape, meaning amplitude and width, frequency, and time as well as the time between pulses, i.e. duty cycle, may be adapted so as to maintain a probe surface temperature between 40 and 45 degrees centigrade. For example, referring to FIG. 10, an exemplary pulsatile waveform is depicted having a period T, a pulse width U, and an amplitude V. Any of these parameters may be changed in order to maintain a probe surface temperature between 40 and 45 degrees centigrade or to achieve a desired power or applied energy per linear centimeter, power or applied energy per square centimeter, or total applied energy. The duty cycle of the pulsatile waveform is defined as the pulse width U, measured in units of time, divided by the waveform period T, measured in units of time.

In any embodiment of the method of the invention, the desired treatment parameters may be any of the following, either alone or in any combination: 1) the administering of between 2000 4000 joules to the treatment area; 2) the administering of between 200-600 joules per linear centimeter to the treatment area; 3) the administering of between 21.068-149.208 joules/cm² to the treatment area; 4) or the administering of between 13.045-310.546 joules/cm² to the treatment area.

In any of the embodiments of the invention, the massaging portion and the light emitting portion may be fabricated from any material that is transmissive at near infrared wavelengths, such as silica glass, plastic, or any other material that is transmissive to laser energy over the defined near infrared wavelength range.

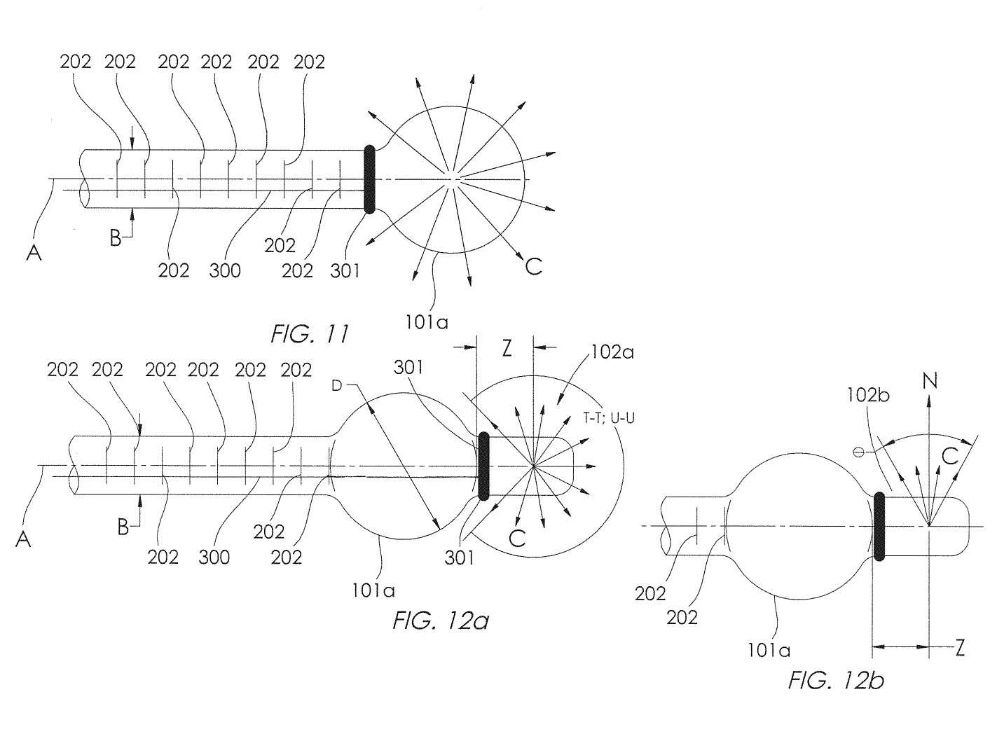

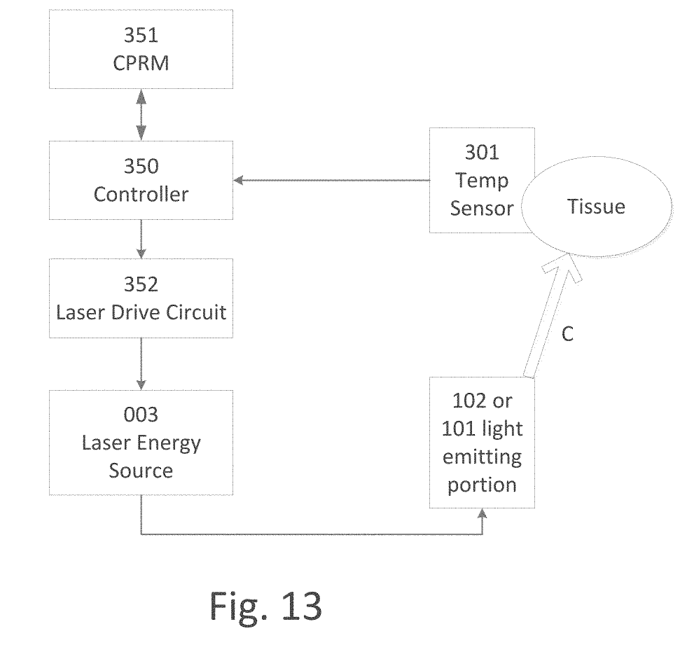

Regarding FIG. 11. The probe light emitting portion and massaging portion 101 may be the same structure, as depicted in FIGS. 3A, 3B, 4A, 4B, 5A and 5B. Likewise, an embodiment of the probe which further comprises a temperature sensor 301 and connecting wiring 300 is depicted in FIG. 11. Temperature sensor 301 may be connected to a controller 350 via wiring 300 as is further described in relation to the exemplary electrical block diagram of FIG. 13. Temperature sensor 301 may comprise any temperature sensor known in the art. For example, temperature sensor 301 may comprise a thermistor, semiconductor device, or any other electrical device that exhibits a change in at least one electrical characteristic proportional to, or in a known relationship to, changes in temperature. Temperature sensor 301 may comprises a thermally conductive element that is in physical contact with at least a portion of the tissue near the light emitting element of the probe, such that when the tissue changes temperature, the at least one electrical characteristic of the temperature probe also changes in a known relationship with the change in tissue temperature. Temperature probe 301 may be in communication with controller 350 (shown in FIG. 13). Controller 350 is adapted to measure the change in the at least one electrical characteristic of the temperature probe, and thereby is able to estimate the temperature of the tissue. Using this information, Controller 350 may then command the laser energy source 003 to either provide more or less power to achieve a particular tissue temperature, or to keep the tissue between a lower and upper limit of temperature. In an alternative embodiment, the temperature sensor may be comprised of an alexandrite fluorescence lifetime fiber optic thermometer in communication with controller 350. Probe body outer dimension B is shown for reference, as is probe longitudinal axis A and laser energy C being emitted by massaging body 101a. In the embodiment shown in FIG. 11, the massaging portion and the light emitting portion are the same structure and may be fabricated from any material that is transmissive at near infrared wavelengths, such as silica glass, plastic, or any other material that is transmissive to laser energy over the defined near infrared wavelength range. Probe body 100 may comprise markings 202 at specific intervals, for example one centimeter spacing, to assist a surgeon with translating the probe over a predetermined linear distance.

Referring now to FIG. 12A, an embodiment of a probe of the invention in which the massaging portion 101a is separate from the light emitting portion 102a is depicted. In this embodiment the probe comprises a body portion 100, a light emitting portion 102a and a massaging portion 101a wherein light emitting portion is transmissive to near infrared laser energy; and wherein the probe is capable of being in optical communication with a source of laser energy 003 such that laser energy is transmissible from the laser source 003, through the body portion and into said light emitting portion 102a. The light emitting portion 102a is capable of emitting the laser energy C into tissue surrounding light emitting portion 102a when said laser source 003 is activated. The massaging portion 101a has an outer dimension D that is larger than the body portion outer dimension B. The probe configuration shown in FIG. 12A may be used to treat any body tissue but is also specifically adapted to emit laser energy C into vaginal tissue. Laser energy C may be emitted in a partially spherical radiation pattern as depicted in angles T-T and U-U in FIG. 6, wherein the spherical pattern is centered around an axis that is coaxial with the longitudinal axis of the probe A. Probe body 100 may comprise markings 202 at specific intervals, for example one centimeter spacing, to assist a surgeon with translating the probe over a predetermined linear distance. As described above, temperature sensor 301 may be in communication with controller 350 for measuring tissue temperature and controlling laser source 003 parameters, such as output power or duty cycle, or both, to achieve a desired tissue temperature range. In the embodiment shown in FIG. 12A, the light emitting portion 102a and the massaging portion 101a are separated by a distance Z as measured from the center of laser energy C emission from the light emitting portion 102a to the nearest surface of massaging portion 101a. An exemplary value for Z is one to two centimeters, but any other desired value for Z may be used. Massaging portion 101a is depicted as being spherically shaped in FIG. 12A, but the embodiments of the probe in which the massaging portion 101 is not the same structure as the light emitting portion may comprise any outer shape (e.g. spherical, conical, trapezoidal, or otherwise) for the massaging portion 101 or light emitting portions 102a.

Referring now to FIG. 12B, an embodiment of a probe of the invention in which the massaging portion 101a is separate from the light emitting portion 102a is depicted. In this embodiment the probe comprises a body portion 100, a light emitting portion 102a and a massaging portion 101a wherein light emitting portion is transmissive to near infrared laser energy; and wherein the probe is capable of being in optical communication with a source of laser energy 003 such that laser energy is transmissible from the laser source 003, through the body portion 100 and into the light emitting portion 102a. The light emitting portion 102a is capable of emitting the laser energy C into tissue surrounding light emitting portion 102a when said laser source 003 is activated. The massaging portion 101a has an outer dimension D that is larger than the body portion outer dimension B. The probe configuration shown in FIG. 12B may be used to treat any body tissue but is also specifically adapted to emit laser energy C into rectal tissue. Laser energy C may be emitted in a partially spherical radiation pattern as depicted in angles in FIGS. 8A and 8B, wherein the spherical pattern is centered around an axis N that is orthogonal to the longitudinal axis of the probe A. Probe body 100 may comprise markings 202 at specific intervals, for example one centimeter spacing, to assist a surgeon with translating the probe over a predetermined linear distance. As described above, temperature sensor 301 may be in communication with controller 350 for measuring tissue temperature and controlling laser source 003 parameters, such as output power or duty cycle, or both, to achieve a desired tissue temperature range. In the embodiment shown in FIG. 12B, the light emitting portion 102a and the massaging portion 101a are separated by a distance Z as measured from the center of laser energy C emission from the light emitting portion 102a to the nearest surface of massaging portion 101a. An exemplary value for Z is one to two centimeters. Massaging portion 101a is depicted as being spherically shaped in FIG. 12B, but the embodiments of the probe in which the massaging portion 101 is not the same structure as the light emitting portion may comprise any outer shape (e.g. spherical, conical, trapezoidal, or otherwise) for the massaging portion 101 or light emitting portions 102a.

Referring now to FIG. 12C, an embodiment of a probe of the invention is depicted in which a plurality of marks 500 are disposed on probe body 100, allowing the invention to calculate the Treatment Surface Area 516 by taking into account the laser energy emitting surface area ESA, 516, of probe 001 and the distance of insertion L into the potential space of a body cavity from body cavity entry point Y, which may be for example a vaginal or rectal opening, to end point Z, which may be for example a vaginal apex or the distal extent of a prostate. Body tissue comprising the potential space X maybe displaced by probe 001 as probe 001 is translated through the potential space resulting in Treatment Surface Area TSA 516, thus massaging the displaced and stretched body tissue. Probe body 100 may be removably attached to handle 002 such that various probes 001 of different sizes and shapes, and having various ESAs, may attached to handle 002. In this manner a treating physician may select s specific size and shape probe 001, having a specific ESA, for a particular patient based on any factor such the estimated depth of the potential and the ability of the body tissue of the patient to stretch and displace around the outer surface of the probe. Once a specific probe 001 has been chosen, it is attached to probe handle 100. Probe 001 may comprise an identifier that identifies the probe. The identifier may be any marking or identifying feature that identifies the probe has having a specific ESA. The identifier may be any marking such an alphanumeric code that is marked on probe 001. In this case the identifier may be communicated to controller 350 by any user input device in communication with controller 350. Alternatively, the identifier may be, for example, a coded indicia disposed on the surface of probe body 100 or on its packaging. The controller may be adapted to receive a probe-identifying signal from a reader adapted to read the coded indicia. The coded indicia may be, for example, a bar code or QR code disposed on an exterior surface of the probe, and the reader may be a bar or QR code reader operatively coupled to the controller. In this case, the bar or QR code reader is used to read the bar or QR code, respectively, that is disposed on probe 001. The bar or QR code information is communicated to controller 350. Still further, probe 001 may comprise an embedded electrical circuit or electronic components that become operatively coupled to controller 350 when probe 001 is attached to handle 002 as depicted in FIG. 12 through wiring in handle 002. The electrical circuit or electronic device may be, for example, any circuit such as a resistor network presenting a predetermined resistance, a logic wiring returning a digital word by providing opens and shorts in a predetermined order; an active electronic device, or any combination or variation of these elements. Probe 001 may also communicate its identifier to controller 350 wirelessly such as through Bluetooth®, Bluetooth LE®, WiFi, optical infrared transceivers, RFID tag and reader operatively coupled to controller 350, or any other wireless means of communication known in the art. In all these embodiments, the probe-identifying information is communicated to controller 350. Controller 350 may then utilize previously stored, or presently entered, ESA information about the specific probe 001 that has been attached to handle 002 to calculate the TSA for a given probe and a given distance of insertion L as follows.

Still referring to FIG. 12C, a plurality of sensors 517 may be arranged along probe body 100 in a pattern that is usable by controller 350 determine the distance of insertion L of probe tip 101 into a body cavity to be treated. In a preferred embodiment, the plurality of sensors 517 are disposed in a linear array, each sensor equally spaced from its adjacent sensor. However it is not necessary that sensors 517 be disposed in an equally-spaced linear array. Each sensor may be in communication with controller 350 via electrical wiring or other conductive means running lengthwise through probe body 100, handle 002, and cable 004. Sensors 517 may comprise temperature sensors, EMG sensors, pH sensors, pressure sensors or light sensors. In the case in which sensors 517 are temperature sensors, the body tissue surrounding the probe tip and body is displaced by the exterior surface of probe tip 101 and probe body 100 when probe tip 101 and probe body 100 are inserted into a body cavity comprising a potential space as sensors 517 sequentially enter the body cavity potential space and thus come into contact with body tissue, thus sequentially raising the temperature of each temperature sensor that enters the body cavity potential space. In this manner, when probe tip 101 and probe body 100 are inserted into a body cavity comprising a potential space, the sensors 517 will sequentially sense a rise in temperature as the probe is inserted. In use, controller 350 communicates with each temperature sensor 517 to determine how many sensors 517 have been inserted into a body cavity comprising a potential space. Likewise, in the embodiment in which sensors 517 are pressure sensors, the body tissue surrounding the probe tip and body is displaced by the exterior surface of probe tip 101 and probe body 100 when probe tip 101 and probe body 100 are inserted into a body cavity comprising a potential space as sensors 517 sequentially enter the body cavity potential space and thus come into contact with body tissue, thus sequentially raising the pressure on each pressure sensor 517 that enters the body cavity potential space. In this manner, when probe tip 101 and probe body 100 are inserted into a body cavity comprising a potential space, the pressure sensors 517 will sequentially sense a rise in pressure as the probe is inserted. In use, controller 350 communicates with each pressure sensor 517 to determine how many sensors 517 have been inserted into a body cavity comprising a potential space. Still further, in the embodiment in which sensors 517 are light sensors, the body tissue surrounding the probe tip and body is displaced by the exterior surface of probe tip 101 and probe body 100 when probe tip 101 and probe body 100 are inserted into a body cavity comprising a potential space as sensors 517 sequentially enter the body cavity potential space and thus come into contact with body tissue, thus sequentially blocking light from being received by each light sensor 517 that enters the body cavity potential space. In this manner, when probe tip 101 and probe body 100 are inserted into a body cavity comprising a potential space, the light sensors 517 will sequentially sense a blockage, or loss, of light as the probe is inserted. In use, controller 350 communicates with each light sensor 517 to determine how many sensors 517 have been inserted into a body cavity comprising a potential space. Likewise, in the embodiment in which the sensors are pH sensor, each sensor that enters a body cavity will measure a change in pH and in the embodiment in which the sensors are EMG sensors, each sensor that enters a body cavity will measure a change in electrical activity. Thus, controller 350 may use the information provided by sensors 517 to determine the distance of insertion L of the probe into the body cavity. Alternatively, a user, such as a treating physician, nurse or assistant, may use markings 500, depicted in exemplary fashion as M1-M7 to observe how far a probe 001 has been inserted into the potential space of a body cavity, and may communicate this information directly to controller 350 using any user input device that is in communication with controller 350.

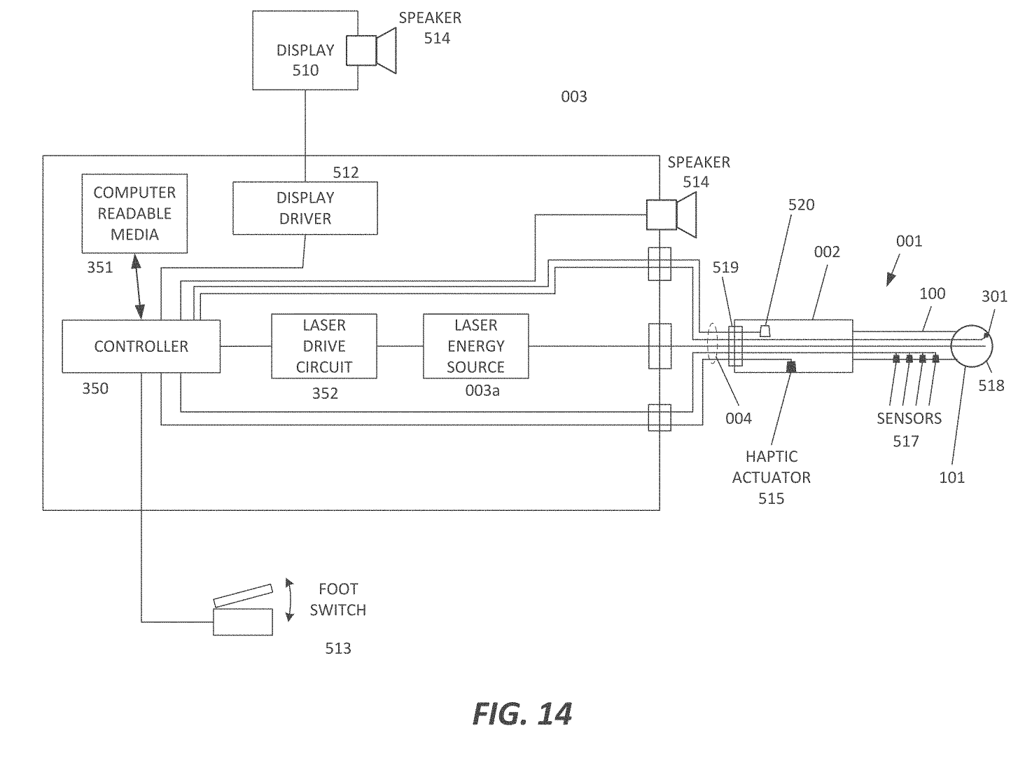

Controller 350 then compares the identification of the probe 001 to pre-stored probe handle in information stored in computer readable memory 351, depicted in FIG. 14, to identify the specific probe 001 in use, and reads from the ESA of probe 001. Using any of the above means, or any other means known in the art, controller 350 is able to determine the area of TSA 516 by calculating the laser emitting surface area ESA of probe body 001 using distance L as determined above.

Referring now to FIG. 13, a tissue temperature controlling feature of an embodiment of the invention is shown. A temperature measuring element 301 may be located on the probe as depicted, by way of example, in FIGS. 11, 12A and 12B. The temperature measuring element 301 may be any element for sensing a temperature and producing a signal proportional to the temperature, or which exhibits a change in electrical characteristic proportional to, or in a known relationship to, changes in temperature. Temperature sensing element 301 may comprise a thermally conductive element that is in physical contact with at least a portion of the tissue near the light emitting element of the probe, such that when the tissue changes temperature, the at least one electrical characteristic of the temperature probe also changes in a known relationship with the change in tissue temperature. Alternatively, temperature sensing element 301 may also comprise a digital thermometer circuit that produces a digital output that is coded to represent the temperature of the measured tissue. Temperature probe 301 may be in communication with controller 350. Controller 350 is adapted to measure the change in the at least one electrical characteristic of the temperature probe, and thereby is able to estimate the temperature of the tissue. As an example, when controller 350 is a device that is adapted to perform functions by the execution of non-transitory computer readable instructions, such instructions may be stored in computer readable media (CRM) 351 that is in communication with controller 350. Controller 350 may read and execute the non transitory instructions stored in CRM 352 to perform the processing of the temperature signal from temperature sensing element 301 Using this information, controller 350 may then command the laser energy source 003 to either provide more or less power to achieve a particular tissue temperature, or to keep the tissue between a lower and upper limit of temperature. This may be done by controller 350 directly controlling the output power or duty cycle of laser energy source 003, or by commanding an intervening laser drive circuit 352 is a controllable circuit adapted to receive a control signal from controller 350 and to control the output power or duty cycle of laser energy source. On activation, laser energy source 003 provides laser energy to the probe through an optically transmissive path, which may be, for example, an optical fiber that is transmissive at near infrared wavelengths. The laser energy is emitted through either the massaging portion 101 or light emitting portion 102 as depicted by arrow C into to the tissue surrounding the probe. Temperature sensing element 301 is in at least temporary physical contact with the tissue near to or surrounding the probe massaging portion. Temperature sensing element 301 provides a signal proportional to the temperature of the tissue as hereinbefore described, and controller 350 may then execute non-transitory computer readable instructions for reading the temperature of said tissue and controlling said laser drive circuit in order to adjust a laser source parameter selected from the group consisting of amplitude and duty cycle in order to maintain said tissue at a temperature between 40° and 45° centigrade, or any other temperature range desired or predetermined.

Referring now to FIG. 14, a block diagram of an embodiment of the invention is depicted. In an embodiment, a laser source of the invention 003 may comprise laser optical source 003a that is characterized by laser output power P in communication with a laser drive circuit 352 to provide electrical power and drive current to laser source 003a. Laser drive circuit 352 may be in communication with controller 350, which may be in communication with non-transitory computer readable media 351. Controller executable instructions for carrying out the steps of the invention may reside in computer readable media 351. Display driver 512 may also be in communication with controller 352 for receiving display video information and commands, and displaying video information, in accordance with the steps of the invention. Display 510 may also comprise a speaker 510 that is in communication with and may receive audio signals from controller 352 for producing audio in accordance with the steps of the invention. Foot switch 513 may be in communication with controller 352 for controlling laser source 003a to an ON state, in which laser energy is transmitted to probe 001 to be transmitted into body tissue through probe massaging portion 101; or to an OFF state, in which no laser energy is transmitted to probe 001 or to probe massaging portion 101. Optical and electrical communication may be provided by cable 004, which may be in communication with source of laser energy 003 and with probe 001. Probe handle 002 and probe massaging portion 101 are called out for reference. Probe handle 002 may comprise haptic actuator 515 which is in communication with controller 352. Probe connector 100 may comprise one or more sensors 517 as more fully described in reference to FIG. 12C. Controller 350 may also be in communication with a foot pedal or foot switch 513 which may be used to command laser source 3a in to an ON state or OFF state as desired by the user.

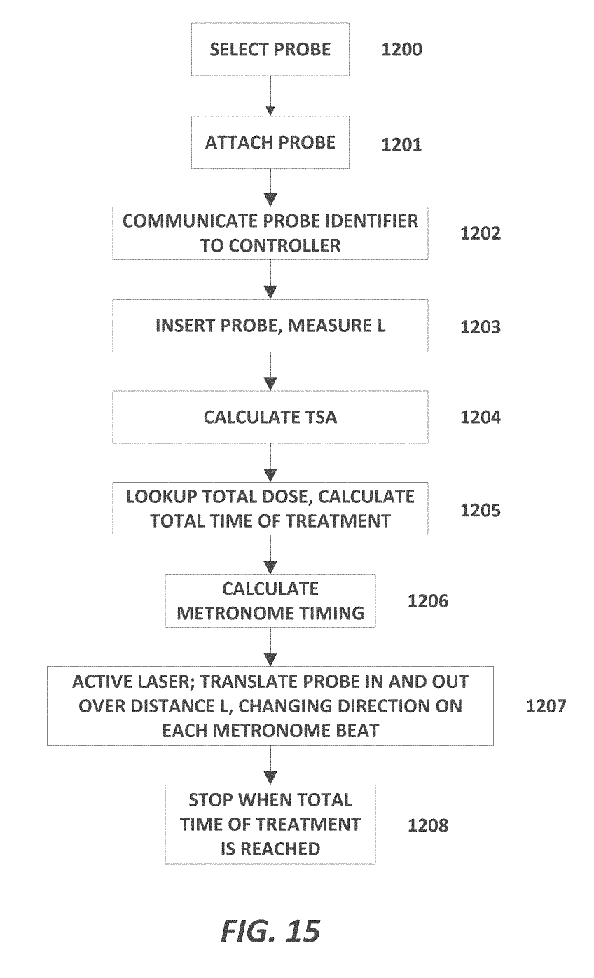

Referring now to FIG. 15, and FIGS. 12c and 14, in an embodiment, the invention also comprises a method for treating any body tissue, including but not limited to treating pelvic pain, chronic prostatitis, or overactive bladder, comprising the steps of: identifying a probe characterized as having a specific ESA that is appropriate to treat a specific patent, step 1200; attaching the selected probe 001 to handle 002 such that probe 001 is in optical communication with a laser energy source 003a characterized by an output power Pstep 1201; communicating a probe identifier to controller 350, step 1202; inserting probe 001 into body cavity comprising body tissue to be treated, which may comprise a potential space, for a total probe translation distance L, measuring L by as hereinbefore described or by any other method, and communicating L to controller 350, step 1203; calculating a total treatment surface area TSA using probe 001 ESA information and distance L information, the calculation preferably but not necessarily being performed by controller 350, step 1204; using a predefined total dose value, determining a total laser ON time T for a pre-determined energy/unit area (J/area), where T=(TSA*J/area)/P, step 1205; using T and L, calculate the timing between metronome beats from a metronome signal selected from the group consisting of an audio signal, a video signal or a haptic signal for signaling when to transition from IN to OUT probe translation and vice versa, step 1206; inserting probe 001 into a body cavity which may be a vagina or a rectum of a patient, activating laser energy source 3a causing laser energy to be emitted by probe 001 through probe laser energy emitting surface area ESA, wherein the laser energy illuminates body tissue as it passes by ESA, the body tissue being aginal tissue in the case in which the probe is inserted into a vagina, or rectal tissue in the case in which the probe is inserted into a rectum, changing from IN to OUT probe translation or vice versa on each metronome beat, step 1207; and continuously translating the probe at a rate of translation along the longitudinal axis of the probe in an IN and OUT motion for period of time T while the probe emits laser energy from the laser energy emitting surface area ESA, step 1208. The movement of probe 001 along distance L causes laser energy emitting surface area ESA to be translated over distance L, resulting in a total treatment surface area TSA

The laser energy may be emitted from probe 001 such that the energy distribution of the laser energy emitted in a plane that is perpendicular to the longitudinal probe axis is non-uniform. The method may further comprise the step of rotating the probe about the longitudinal probe axis In an embodiment, such rotation of the probe may occur as the probe is translated in the IN and OUT motion.

The distance L may be determined by measuring or recording the distance traveled by probe 001 when the probe 001 is translated from a vaginal opening to a desired distal point of treatment, such as the vaginal apex, in the case of intravaginal use; or when the probe is translated from the opening of an anus to a desired distal point of treatment, in the case of rectal use. The probe may further be defined as comprising a probe body having a plurality of gradation markings, and wherein the step of determining L is further defined as observing the number of gradation markings that pass through the vaginal opening as the probe is translated from the vaginal opening to the desired distal point of treatment. The probe may further be defined as comprising a probe body having a plurality of gradation markings, wherein the step of determining d is further defined as determining the number of gradation markings that pass through the anus as the probe is translated from the anus to the desired distal point of treatment.

The probe may further be defined as comprising a probe body having at least one sensor, and wherein the step of determining d is further defined as sensing the distance of translation of the probe as it travels from the vaginal opening to the desired distal point of treatment in the case of intravaginal use, or as it travels from the anus to the desired distal point of treatment in the case of rectal use. The at least one sensor may be selected from the group consisting of a temperature sensor, a pressure sensor, an electrical conductivity sensor, a pH sensor and a light sensor.

The probe translation may further defined as a continuous IN and OUT movement of the probe within the potential space over distance d, and wherein the rate of translation of the probe is determined by a periodic metronome signal that provides a cadence. The metronome signal may be signal, with each beat, a change in direction from IN to OUT motion, and vice versa. The metronome signal may be selected from the group consisting of an audio signal, a video signal or a haptic signal. The rate of translation may be between 2.5 and 60 millimeters per second. The translation is may be a continuous IN and OUT motion.

The desired distal point of treatment may be the vaginal apex in the case of vaginal use. The desired distal point of treatment may be defined as the most distal extent of the prostate in the case of rectal use. The translation of probe 001 in the potential space may cause massaging of vaginal tissue in the case of intravaginal use, or rectal tissue in the case of rectal use, by the displacement of vaginal or rectal tissue along probe outer surfaces as probe 001 is translated through a potential space.

The light energy density applied to the body tissue in any of the methods of the invention, in J/area, may be between 13.045-310.546 joules/cm2 or any sub-range within this range.

What is claimed is:

1. A method for treating body tissue, comprising:
providing a source of laser energy characterized by a laser power P;
providing a probe having a longitudinal axis capable of emitting laser energy to vaginal tissue or rectal tissue, said probe in optical communication with said source of laser energy, said probe being characterized as having a predetermined outer surface laser energy emitting surface area ESA;
inserting said probe into a potential space of a vagina or rectum of a patient such that at least a portion of said probe outer surface is in physical contact with a portion of said vaginal or said rectal tissue;
determining a length of intravaginal or intrarectal probe travel L, wherein movement of said probe along distance L causes laser energy emitting surface area ESA to be translated over distance L, resulting in a total treatment surface area TSA;
determining a total laser ON time T for a pre-determined energy/unit area (J/area),
where
T=(TSA*J/area)/P;
inserting said probe into a vagina or a rectum of a patient;
causing laser energy to be emitted by said probe thru outer surface laser energy emitting surface area ESA, wherein said laser energy illuminates said vaginal tissue in the case in which the probe is inserted into a vagina, or illuminates said rectal tissue in the case in which said probe is inserted into a rectum; and
translating said probe at a rate of translation along said longitudinal axis of said probe for period of time T while said probe emits said laser energy from said laser energy emitting surface area ESA.

2. The method of claim 1, wherein said laser energy is emitted from said probe such that the energy distribution of said laser energy emitted in a plane that is perpendicular to said longitudinal probe axis is non-uniform, and further comprising the step of rotating said probe about said longitudinal probe axis.

3. The method of claim 1, wherein L is determined by recording the distance traveled by said probe when said probe is translated from a vaginal opening to a desired distal point of treatment, in the case of intravaginal use; or when said probe is translated from the opening of an anus to a desired distal point of treatment, in the case of rectal use.

4. The method of claim 3, wherein said probe is further defined as comprising a probe body having a plurality of gradation markings, and wherein the step of determining L is further defined as observing the number of gradation markings that pass through the vaginal opening as the probe is translated from the vaginal opening to the desired distal point of treatment.

5. The method of claim 3, wherein said probe is further defined as comprising a probe body having a plurality of gradation markings, and wherein the step of determining L is further defined as determining the number of gradation markings that pass through the anus as the probe is translated from the anus to the desired distal point of treatment.

6. The method of claim 3, wherein said probe is further defined as comprising a probe body having at least one sensor, and wherein the step of determining L is further defined as sensing the distance of translation of the probe as it travels from the vaginal opening to the desired distal point of treatment in the case of intravaginal use, or as it travels from the anus to the desired distal point of treatment in the case of rectal use.

7. The method of claim 6, wherein said at least one sensor is selected from the group consisting of a temperature sensor, a pressure sensor, an electrical conductivity sensor, a pH sensor and a light sensor.

8. The method of claim 1, wherein said translation is further defined as a continuous back and forth movement of the probe within said potential space over distance L, and wherein said rate of translation of said probe is determined by a periodic metronome signal that provides a cadence.

9. The method of claim 8, wherein said metronome signal is selected from the group consisting of an audio signal, a video signal or a haptic signal.

10. The method of claim 1, wherein said rate of translation is defined as being between 2.5 and 60 millimeters per second.

11. The method of claim 1, wherein said translation is a continuous in and out motion.

12. The method of claim 3, wherein the desired distal point of treatment is the vaginal apex in the case of vaginal use.

13. The method of claim 3, wherein the desired distal point of treatment is defined as the most distal extent of the prostate in the case of rectal use.

14. The method of claim 1, wherein said translation causes massaging of vaginal tissue in the case of intravaginal use, or rectal tissue in the case of rectal use, by the displacement of vaginal or rectal tissue along probe outer surfaces as said probe is translated.

15. The method of claim 1, wherein said J/area is between 13.045-310.546 joules/cm², inclusive.

16. The method of claim 11, wherein said J/area is between 13.045-310.546 joules/cm², inclusive.

17. The method of claim 14, wherein said J/area is between 13.045-310.546 joules/cm², inclusive.

18. A device for treating body tissue, comprising:
a probe comprising a body portion and a massaging portion;
wherein each of said probe and massaging portion have an outer dimension and are transmissive to said laser energy; and
wherein said probe is capable of being in optical communication with a source of laser energy such that said laser energy is transmissible from said source of laser energy, through the body portion and into the massaging portion;
wherein said massaging portion is capable of emitting said laser energy into vaginal tissue surrounding said massaging portion when said probe is inserted a vagina of a user, or rectal tissue surrounding said massaging portion when said probe is inserted a rectum of a user, when said laser source is activated; and
wherein said massaging portion has an outer dimension that is larger than said body portion; and
wherein said body portion comprises at least one indicator for determining a distance d, wherein d represents the distance said massaging portion has been inserted into the vagina or rectum of a user.

19. The device of claim 18, wherein said at least one indicator is a marking visible on an exterior surface of said probe body.

20. The device of claim 19, wherein said at least one indicator is further defined as a plurality of markings.

21. The device of claim 18, wherein said at least one indicator provides an indication of the distance said probe is inserted into a vagina or rectum of a user.

22. The device of claim 19, wherein said marking provides an indication of the distance said probe is inserted into a vagina or rectum of a user.

23. The device of claim 20, wherein said markings provide an indication of the distance said probe is inserted into a vagina or rectum of a user.

24. The device of claim 20, wherein said plurality of markings is further defined by each of said markings of said plurality of markings being equidistant from adjacent markings by a predetermined distance.

25. The device of claim 24, wherein said predetermined distance is one centimeter.

26. A system for treating body tissue, comprising:
a probe comprising a body portion and a massaging portion operatively coupled to a controllable source of laser energy by an optically transmissive fiber;
a controller operatively coupled to said source of laser energy;
wherein each of said probe and massaging portion have an outer dimension and are transmissive to said laser energy; and
wherein said probe is in optical communication with said source of laser energy such that said laser energy is transmitted from said source of laser energy through the massaging portion and into body tissue surrounding said massaging portion through a predetermined outer surface laser energy emitting surface area ESA when said controllable source of laser energy is activated;
wherein said massaging portion has an outer dimension that is larger than said body portion, and wherein said massaging portion is further characterized as having a outer surface laser energy emitting surface area ESA; and
wherein said body portion comprises at least one indicator for determining a distance d, wherein d represents the distance said massaging portion has been inserted into the vagina or rectum of a user.

27. The system of claim 26, wherein said at least one indicator is further defined as comprising a thermal sensor that produces an output signal proportional to the temperature of the environment surrounding the thermal sensor, and wherein said thermal sensor is operatively coupled to said controller.

28. The system of claim 27, wherein said at least one indicator is further defined as a plurality of thermal sensors.

29. The system of claim 28, wherein said controller is adapted to execute computer readable instructions for receiving the output signal of each thermal sensor to determine the temperature of the environment surrounding each thermal sensor.

30. The system of claim 29, wherein said controller is further adapted to execute computer readable instructions for determining the distance d said probe is inserted into the vagina or rectum of a user using the determined temperature of the environment surrounding each thermal sensor to determine how many thermal sensors have been inserted into the vagina or rectum of a user.

31. The system of claim 30, wherein said controller is further adapted to execute computer readable instructions for controlling said controllable source of laser energy to be active for a total ON time T for said controllable source of laser energy using the formula T=(TSA*J/area)/P wherein P is defined as the laser power P, wherein movement of said probe along distance d causes laser energy emitting surface area ESA to be translated over distance d, resulting in a total treatment surface area TSA, and said J/area is a predetermined energy per unit area for irradiating body tissue in contact with said probe laser energy emitting surface area A.

32. The system of claim 31, wherein said probe further comprises an identifier that identifies ESA for said probe.

33. The system of claim 32, wherein said identifier is a coded indicia, and wherein said controller is further adapted to receive a probe-identifying signal from a reader adapted to read said coded indicia.

34. The system of claim 33, wherein said coded indicia is a bar code disposed on an exterior surface of said probe, and wherein said reader is a bar code reader operatively coupled to said controller.

35. The system of claim 33, wherein said identifier is an electrical or electronic device operatively coupled to said controller.

36. The system of claim 25, wherein said at least one indicator is further defined as comprising an EMG sensor that produces an output signal proportional to the electric potential of body tissue surrounding the EMG sensor, and wherein said EMG sensor is operatively coupled to said controller.

37. The system of claim 36, wherein said at least one indicator is further defined as a plurality of EMG sensors.

38. The system of claim 37, wherein said controller is adapted to execute computer readable instructions for receiving the output signal of each EMG sensor to determine the electric potential of the body tissue surrounding each thermal sensor.

39. The system of claim 38, wherein said controller is further adapted to execute computer readable instructions for determining the distance d said probe is inserted into the vagina of a user using the determined electric potential of the body tissue surrounding each EMG sensor to determine how many EMG sensors have been inserted into the vagina or rectum of a user.

40. The system of claim 39, wherein said controller is further adapted to execute computer readable instructions for controlling said controllable source of laser energy to be active for a total ON time T for said controllable source of laser energy using the formula T=(TSA*J/area)/P wherein P is defined as the laser power P, wherein movement of said probe along distance d causes laser energy emitting surface area A to be translated over distance d, resulting in a total treatment surface area TSA, and said J/area is a predetermined energy per unit area for irradiating body tissue in contact with said laser energy emitting surface area A.

41. The system of claim 36, wherein said at least one EMG sensor is defined as a plurality of EMG sensors.

42. The system of claim 41, wherein said probe further comprises an identifier that identifies A for said probe.

43. The system of claim 42, wherein said identifier is a coded indicia, and wherein said controller is further adapted to receive a probe-identifying signal from a reader adapted to read said coded indicia.

44. The system of claim 43, wherein said coded indicia is a bar code disposed on an exterior surface of said probe, and wherein said reader is a bar code reader operatively coupled to said controller.

45. The system of claim 42, wherein said identifier is an electrical or electronic device operatively coupled to said controller.

46. The system of claim 25, wherein said at least one indicator is further defined as comprising at least one optical sensor that produces an output signal proportional to the intensity of light energy illuminating the optical sensor, and wherein said at least one optical sensor is operatively coupled to said controller.

47. The system of claim 46, wherein said at least one indicator is further defined as a plurality of optical sensors.

48. The system of claim 47, wherein said controller is adapted to execute computer readable instructions for receiving the output signal of each optical sensor to determine intensity of light energy illuminating each optical sensor.

49. The system of claim 48, wherein said controller is further adapted to execute computer readable instructions for determining the distance d said probe is inserted into the vagina of a user using the determined intensity of light energy illuminating each optical sensor to determine how many optical sensors have been inserted into the vagina or rectum of a user.

50. The system of claim 49, wherein said controller is further adapted to execute computer readable instructions for controlling said controllable source of laser energy to be active for a total ON time T.

51. The system of claim 48, wherein said at least one optical sensor is defined as a plurality of optical sensors.

52. The system of claim 49, wherein said probe further comprises an identifier that identifies ESA for said probe.

53. The system of claim 52, wherein said identifier is a coded indicia, and wherein said controller is further adapted to receive a probe-identifying signal from a reader adapted to read said coded indicia.

54. The system of claim 53, wherein said coded indicia is a bar coded disposed on an exterior surface of said probe, and wherein said reader is a bar code reader operatively coupled to said controller.

55. The system of claim 52, wherein said identifier is an electrical or electronic device operatively coupled to said controller.

56. The method of claim 30, wherein said J/area is between 13.045-310.546 joules/cm², inclusive.

57. The method of claim 41, wherein said J/area is between 13.045-310.546 joules/cm², inclusive.

58. A system for treating body tissue, comprising:
a probe comprising a body portion, a massaging portion and a handle portion, said probe operatively coupled to a controllable source of laser energy by an optically transmissive fiber;
a controller operatively coupled to said source of laser energy;
wherein each of said probe and massaging portion have an outer dimension and are transmissive to said laser energy; and
wherein said probe is in optical communication with said source of laser energy such that said laser energy is transmitted from said source of laser energy, through the body portion, through the massaging portion; and into tissue in contact with said massaging portion when said controllable source of laser energy is activated;
wherein said massaging portion has an outer dimension that is larger than said body portion, and wherein said massaging portion is further characterized as having a predetermined outer surface laser energy emitting surface area ESA;
wherein said body portion comprises at least one indicator for determining a distance L, wherein L represents the distance a distal end of probe has been inserted into the vagina or rectum of a user; and
wherein said handle portion remains substantially outside a user's vagina when said massaging portion has been inserted into a user's vagina to the extent of contacting the user's vaginal apex, or when said massaging portion has been inserted into a user's rectum to the extent that the probe reaches a distal end of the user's prostate; and
wherein said handle comprises a haptic actuator operatively coupled to said controller.

59. The system of claim 58, wherein said haptic actuator is disposed on an exterior surface of said handle such that it contacts a user's hand when said user grips the probe handle.

60. The system of claim 59, wherein said controller is adapted to activate said haptic actuator at time intervals for signaling to a treating physician when to transition from an IN motion to an OUT motion and vice versa, wherein, in the case of intravaginal use, said IN motion is defined as moving said probe massaging portion in a direction towards the vaginal apex, and wherein said OUT motion is defined as moving said probe massaging portion in a direction away from the vaginal apex; or, in the case of rectal use, said IN motion is defined as moving said probe massaging portion in a direction towards a distal margin of a patient's prostate, and wherein said OUT motion is defined as moving said probe massaging portion in a direction away from a distal margin of a patient's prostate.

61. The system of claim 60, wherein said time intervals are each of equal magnitude, and wherein the magnitude of said time intervals is determined so as to provide a predetermined rate of translation of said probe over distance L.

62. A system for treating body tissue, comprising:
a probe comprising a body portion, a massaging portion and a handle portion, said probe operatively coupled to a controllable source of laser energy by an optically transmissive fiber;
a controller operatively coupled to said source of laser energy;
wherein each of said probe and massaging portion have an outer dimension and are transmissive to said laser energy; and
wherein said probe is in optical communication with said source of laser energy such that said laser energy is transmitted from said source of laser energy, through the body portion, through the massaging portion; and into tissue surrounding said massaging portion when said controllable source of laser energy is activated;
wherein said massaging portion has an outer dimension that is larger than said body portion, and wherein said massaging portion is further characterized as having a predetermined outer surface laser energy emitting surface area ESA;
wherein said body portion comprises at least one indicator for determining a distance d, wherein d represents the distance a distal end of said probe has been inserted into the vagina of a user, or when a distal end of said probe has been inserted into a user's rectum to the extent that the probe reaches a distal end of the user's prostate; and
wherein said handle portion remains substantially outside a user's vagina when, in the case of intravaginal use, said massaging portion has been inserted into a user's vagina to the extent of contacting the user's vaginal apex; or, in the case of intrarectal use, a distal end of said massaging portion has been inserted into a user's rectum to the extent of reaching a distal margin of a patient's prostate, and
wherein said controller is operatively coupled to a visual display.

63. The system of claim 62, wherein said controller is adapted to provide visual indicators at time intervals for signaling to a treating physician when to transition from an IN motion to an OUT motion and vice versa, wherein, in the case of intravaginal use, said IN motion is defined as moving said probe massaging portion in a direction towards the vaginal apex, and wherein said OUT motion is defined as moving said probe massaging portion in a direction away from the vaginal apex; or, in the case of rectal use, said IN motion is defined as moving said probe massaging portion in a direction towards a distal margin of a patient's prostate, and wherein said OUT motion is defined as moving said probe massaging portion in a direction away from a distal margin of a patient's prostate.

64. The system of claim 63, wherein said time intervals are each of equal magnitude, and wherein the magnitude of said time intervals is determined so as to provide a predetermined rate of translation of said probe over distance L.

65. A system for treating body tissue, comprising:
a probe comprising a body portion, a massaging portion and a handle portion, said probe operatively coupled to a controllable source of laser energy by an optically transmissive fiber;
a controller operatively coupled to said source of laser energy;
wherein each of said probe and massaging portion have an outer dimension and are transmissive to said laser energy; and
wherein said probe is in optical communication with said source of laser energy such that said laser energy is transmitted from said source of laser energy, through the body portion, through the massaging portion; and into tissue surrounding said massaging portion when said controllable source of laser energy is activated;
wherein said massaging portion has an outer dimension that is larger than said body portion, and wherein said massaging portion is further characterized as having a predetermined outer surface laser energy emitting surface area ESA;
wherein said body portion comprises at least one indicator for determining a distance L, wherein L represents the distance said a distal end of said probe has been inserted into the vagina or rectum of a user; and
wherein said handle portion remains substantially outside a user's vagina when said massaging portion has been inserted into a user's vagina to the extent of contacting the user's vaginal apex, or when said massaging portion has been inserted into a user's rectum to the extent that the probe reaches a distal end of the user's prostate; and
wherein said controller is operatively coupled to an audio actuator.

66. The system of claim 65, wherein said controller is adapted to provide audio indicators at time intervals for signaling to a treating physician when to transition probe translation from an IN motion to an OUT motion and vice versa, wherein, in the case of intravaginal use, said IN motion is defined as moving said probe massaging portion in a direction towards the vaginal apex, and wherein said OUT motion is defined as moving said probe massaging portion in a direction away from the vaginal apex; or, in the case of rectal use, said IN motion is defined as moving said probe massaging portion in a direction towards a distal margin of a patient's prostate, and wherein said OUT motion is defined as moving said probe massaging portion in a direction away from a distal margin of a patient's prostate.

67. The system of claim 66, wherein said audio indicator is a beep.

68. The system of claim 67, wherein said time intervals are each of equal magnitude, and wherein the magnitude of said time intervals is determined so as to provide a predetermined rate of translation of said probe over distance L.
